(19) United States
(12) Patent Application Publication
(10) Pub. No.: US 2009/0169286 A1
(43) Pub. Date: Jul. 2, 2009

(54) DEVICE FOR APPLYING A SPREADABLE COMPOSITION TO A PART OF THE HUMAN OR ANIMAL BODY

(75) Inventors: Jan Philip Herweijer, Amsterdam (NL); Marlies van Dullemen, Leiden (NL); Eleonora Doucet-Liem, Leiden (NL); Jan Hendricus Witte, Aerdenhout (NL)

Correspondence Address: BRYAN CAVE POWELL GOLDSTEIN, ONE ATLANTIC CENTER FOURTEENTH FLOOR, 1201 WEST PEACHTREE STREET NW, ATLANTA, GA, 30309-3488, US

(73) Assignee: JPH INVEST B.V., Amsterdam (NL)

(21) Appl. No.: 12/281,213

(22) PCT Filed: Mar. 1, 2007
(86) PCT No.: PCT/NL07/50083

§ 371 Date: Dec. 1, 2008

(30) Foreign Application Priority Data
Mar. 2, 2006 (NL) 2000018

(51) Int. Cl. A61M 35/00; A45D 34/04

(52) U.S. Cl. 401/119; 604/2; 401/145; 401/157

(57) ABSTRACT

A device for applying a spreadable composition to a part of the human or animal body, wherein the device comprises a container containing the spreadable composition, an applicator for the spreadable composition and means for urging the spreadable composition out of the container and into contact with the applicator, provided with positioning means connected to the container for positioning the applicator in a position in which composition urged out of the container can be brought into contact with the applicator.

### PRIORITY CLAIM

This patent application is a U.S. National Phase of International Patent Application No. PCT/NL2007/050083, filed Mar. 1, 2007, which claims priority to Netherlands Patent Application No. 2000018, filed Mar. 2, 2006, the disclosures of which are incorporated herein by reference in their entirety.

### FIELD

The present disclosure relates to a device for applying a spreadable composition to a part of the human or animal body, wherein the device comprises a container containing the spreadable composition, an applicator for the spreadable composition and means for urging the spreadable composition out of the container and into contact with the applicator.

### BACKGROUND

For purposes of the present disclosure, a spreadable composition is, for instance, an ointment, a gel or a cream. Such a device is known in the form of the tube and a wad of cotton, wherein the tube comprises a spreadable composition. By squeezing the tube, a quantity of the spreadable composition can be placed on the wad of cotton and can then be applied to a part of the body.

The application of the spreadable substance to the wad of cotton is usually accompanied by messiness, and the use of this prior art device requires the skill necessary to avoid messiness.

In order to obviate the problems stated hereinabove, the present disclosure provides positioning means connected to the container for positioning the applicator in a position in which the composition urged out of the container can be brought into contact with the applicator.

As a result of these measures, the process of arranging the spreadable substance on the applicator can be much better controlled thus avoiding messiness.

The same effects are achieved by a method for applying a spreadable composition to a part of the human or animal body, the method comprising carrying the spreadable composition out of a container and into contact with an applicator and applying the spreadable composition from the applicator onto the part of a body, wherein the applicator is placed by positioning means connected to the container into a position in which the composition urged from the container can be brought into contact with the applicator.

### SUMMARY

The present disclosure describes several exemplary embodiments of the present invention.

One aspect of the present disclosure provides a device for applying a spreadable composition to a part of the human or animal body, the device comprising a) a container containing the spreadable composition; b) an applicator for the spreadable composition; c) means for urging the spreadable composition out of the container and into contact with the applicator; and d) positioning means connected to the container for positioning the applicator in a position in which the composition urged out of the container can be brought into contact with the applicator.

Another aspect of the present disclosure provides a method for applying a spreadable composition to a part of the human or animal body, the method comprising a) carrying the spreadable composition out of a container and into contact with an applicator; and b) applying the spreadable composition from the applicator onto the part of a body, wherein the applicator is placed by positioning means connected to the container into a position in which the composition urged from the container can be brought into contact with the applicator.

According to one exemplary embodiment, the container is provided with an outflow opening and the positioning means are adapted to position the applicator in a position in the vicinity of the outflow opening, in the direction of movement of the spreadable composition leaving the outflow opening.

This exemplary embodiment avoids the use of guide means for guiding the spreadable composition from the outflow opening to the applicator, which simplifies the structure of the device and greatly limits cleaning operations. The importance of cleaning is noted herein. The applicator is usually used on and in parts of the body wherein the danger of infection is considerable. Being able to easily clean the applicator and those parts of the container which come into contact with the applicator is of the greatest importance. This exemplary embodiment also makes it possible for the applicator to be used more than once.

Yet another exemplary embodiment provides a container whose internal volume can be reduced by external forces acting on the container. The process of applying the spreadable composition to the applicator can hereby be controlled better.

In a simple and inexpensive exemplary embodiment, the container is formed by a tube.

In order to simplify the process of arranging the spreadable composition on the applicator for the user, which is, for instance, important for users with limitations, it is desirable when the container has a rigid outer wall and when the container comprises a propellant gas in addition to the spreadable composition.

A technically optimal structure is obtained when the container is provided with an internal movable wall, on one side of which the spreadable composition is present and on the other side of which the propellant gas is present. This exemplary embodiment makes the choice of a propellant gas easier.

Yet another exemplary embodiment provides a container having a valve which can be operated by the applicator positioned by the positioning means. Operation is greatly simplified hereby and the chance of messiness is reduced.

Messiness when arranging the spreadable substance on the applicator is precluded when the valve can only be operated by an applicator positioned by the positioning means.

In order to prevent the spreadable composition from being absorbed into the applicator, which would be possible with the use of a applicator manufactured from a porous material such as wadding or foam, it is desirable when the applicator is provided with a core with a closed, i.e., non-porous, outer wall, wherein the core is adapted for contact with the spreadable composition. The closed outer wall of the core will prevent the spreadable composition from being drawn into the applicator preventing the spreadable composition from being applied at the desired location. The closed surface of the applicator moreover provides the option of cleaning and re-use thereof.

A further exemplary embodiment provides a core that has a first and second part of a mutually different form, wherein both parts can be placed in the positioning means and both parts are adapted to come into contact with the spreadable composition. The use of parts of mutually differing form provides the option of making a choice as to the part on which the spreadable substance is arranged. The user can let this choice depend on the nature of the location on the body where the spreadable substance must be applied, such as a body cavity. The spreadable substance is then arranged on the first part while the other part is used to grip the core.

The construction of the core is simplified when the core is rotation-symmetrical.

The options for use of the applicator increase when a sleeve extending around the core is also arranged in addition to the core. This sleeve can then be used to hold the spreadable substance securely between the core and the sleeve during transport between the container and the skin. For this purpose the sleeve is preferably formed such that it encloses a space between the sleeve and the core. This space can then be filled with the spreadable substance. The sleeve can be arranged around either the first or the second part of the core, although the sleeve can also be arranged around both parts of the core.

This is achieved by a method wherein the spreadable composition is carried out of the container into a space formed in the applicator.

According to another exemplary embodiment, the sleeve is provided with openings. These openings are used to apply the spreadable composition. The applicator is particularly suitable for applying the spreadable composition in body cavities. This is because the spreadable composition exits not only at the tip but also on the sides of the applicator so that the composition is well distributed over the side surfaces of the body cavities.

These advantages are also achieved in a method wherein the spreadable substance is carried at least partially through the filling opening into and out of the space.

When the container is a container provided with a propellant gas, a method is preferably used wherein the spreadable composition flows out of the container into the space formed in the applicator through operation of a valve of the container by the applicator.

In order to facilitate filling and emptying the space, it is desirable for the sleeve to be movable in an axial direction relative to the core. This is because it becomes possible, in combination with an appropriate design of the core and the sleeve, to vary the volume of the space between the core and the sleeve which can be used to urge out the spreadable substance, particularly when the spreadable substance is being applied.

The same effect is achieved by a method wherein the spreadable composition is urged from the applicator to the body by reducing the volume of the cavity formed in the applicator.

In order to be able to drive or operate the relative movement of the sleeve and the core by hand, it is desirable when the sleeve is provided with a collar extending substantially in a radial direction. This collar can then be gripped with the fingers.

Urging the spreadable substance out of the space between the core and the cavity is facilitated when the sleeve is adapted to be closed internally by the core over a substantial part of its path of movement. The core then functions as a piston inside the sleeve functioning as a cylinder.

The structure of the applicator is simplified when the interior of the sleeve is cylindrical. The core can slide reciprocally in the sleeve in the manner of a piston in a cylinder.

For some applications it can be desirable to give the sleeve a conical form on the outside. It is then possible to give the inner side of the sleeve a cylindrical form so that the measures stated hereinabove can be applied. It is also possible to make use of a largely conical cavity, wherein the sleeve is manufactured from flexible material such that the sleeve can be deformed such that the sleeve is sealed by the cylindrical core.

Filling of the applicator is moreover facilitated when the sleeve is provided on its end remote from the core with a filling opening for filling the space between the core and the sleeve with the spreadable composition. In order to prevent a large part of the spreadable composition flowing out via this filling opening during application, it is desirable when the filling opening is not too large.

It is, however, also possible for the core to be hollow and for the core to be provided with a filling opening for filling the space between the core and the sleeve with the spreadable composition. The hollow space of the core can be placed into connection with the valve of the container. It is desirable for the valve to be operated by the core, for instance, the core engages the valve.

The filling opening is adapted to fill the space between core and sleeve, but this filling opening can also be used to urge the spreadable composition out during application.

In order to facilitate the dosaging of the spreadable composition, a scale division is arranged on the core. This indicator can be used when filling the space. The core does after all protrude from the sleeve, so that a scale division arranged on the core is easy to read.

A method wherein the volume of the space is read during filling of the space between core and sleeve also results in these advantages.

When the sleeve and the core are adapted to displace the core relative to the sleeve when the space between the core and the sleeve is filled through the filling opening arranged in the sleeve, the core can be moved outward in the manner of a piston during filling so as to provide space for the spreadable composition.

The filling procedure of the applicator is greatly simplified when the sleeve is adapted to operate the valve of the container. In order to fill the applicator, it is then only necessary to press the sleeve with the fingers until a sufficient quantity of the spreadable composition has entered the applicator.

The transfer between the sleeve and the valve is simplified when the sleeve is adapted to engage on an operating element of the valve.

In order to prevent lateral movements of the applicator, it is desirable for the positioning means to be adapted to fix the sleeve by means of a snap connection.

The operation of the filling process is facilitated when the snap connection is adapted to allow a movement of the sleeve in an axial direction relative to the positioning means.

It is possible to design both the core and the sleeve, or each individually, for once-only use. It is, however, desirable to design at least one of the sleeve or the core for repeated use. Both parts, but, in particular, the core, can be readily cleaned after use. In some cases, it can be desirable to replace the sleeve after each use since the sleeve cannot be cleaned quite so well due to the presence of the openings.

When the cavity in the sleeve is filled and the applicator must be removed in its entirety, there is a danger, due to the closure between the valve and the sleeve, of an underpressure being created in the sleeve which forces the content of the cavity outward while the core moves inward. In order to prevent this problem, a further exemplary embodiment arrests the core in the sleeve. Although an underpressure is still developed here, the underpressure is only developed until the filling opening has been released from the valve.

In order to perform this arrest as easily as possible, it is recommended that at least a part of the sleeve is compressible and that the core is arrested in the sleeve when the sleeve is compressed.

This compressibility can be achieved by providing the sleeve with notches on either side of its cavity.

An important use of the device relates to the application of the spreadable composition in a body cavity, such as, for example, the pharynx, the ears, the nostrils, the armpits or the vagina.

A further use relates to the application of the spreadable substance in the anus.

The present disclosure also relates to a container which is adapted as a container in a device as described hereinabove, and the present disclosure also relates to an applicator which is adapted as an applicator in a device as described hereinabove.

### BRIEF DESCRIPTION OF THE DRAWINGS

Various aspects of the present disclosure are described hereinbelow with reference to the accompanying figures.

FIG. 1a is a cross-sectional view of a first exemplary embodiment of the present disclosure with an administering core in a first position;

FIG. 1b is a cross-sectional view of the exemplary embodiment of FIG. 1A with an administering core in a second position;

FIG. 2 is a cross sectional view of a second exemplary embodiment;

FIG. 3a is a cross-sectional view of a third exemplary embodiment in a first position;

FIG. 3b is a cross-sectional view of a third exemplary embodiment in a second position;

FIG. 3c is a cross-sectional view of a third exemplary embodiment in a third position;

FIG. 3d is a cross-sectional view of a third exemplary embodiment in a fourth position;

FIG. 3e is a cross-sectional view of a third exemplary embodiment in a fifth position;

FIG. 3f is a cross-sectional view of a third exemplary embodiment in a sixth position;

FIG. 4 is a cross-sectional view of a variant of the third exemplary embodiment; and

FIG. 5 is a cross-sectional view of a fourth exemplary embodiment.

### DETAILED DESCRIPTION

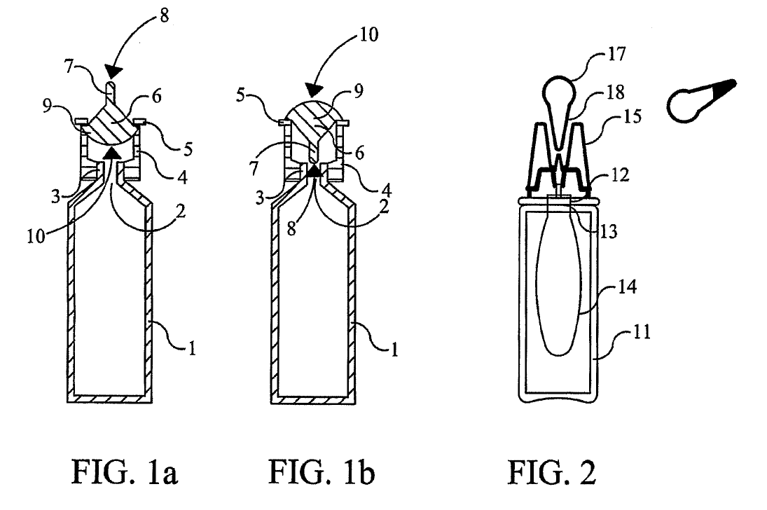

FIGS. 1a and 1b show a tube 1 which serves as a container and which is filled with a spreadable substance, such as a cream or an ointment. Tube 1 is provided with an opening 2 which is enclosed by an annular element 3 which is provided on its outside with a screw thread. A positioning element in the form of a cylinder 4 is screwed onto the screw thread. The positioning element is provided for this purpose with an opening having an internal screw thread. On the side remote from the opening, the positioning element is provided with a positioning ring 5. This positioning ring 5 serves for positioning an applicator 6.

The applicator 6 shown in FIG. 1a comprises a substantially cylindrical part 7 which is provided with a rounded top 8 and a substantially conical part 9, which is provided with a round base 10 and the top of which transposes into cylindrical part 7. Applicator 6 is adapted to apply the spreadable material in the form of an ointment or a cream to the body. The form of the applicator depends on the place on the body where the ointment or cream is applied, such as a body cavity.

In use of this exemplary embodiment, the user places the applicator with the desired administering side on the positioning device, wherein the position of the applicator is determined by positioning ring 5. The user then presses tube 1, whereby a reduction in the volume of the content of the tube occurs and spreadable composition flows out of opening 2 of tube 1. The composition enters the interior of the positioning device and comes into contact with the spherical surface 10 of applicator 6. The applicator is then removed and used to apply the ointment or cream at the desired location on or in the body.

As shown in FIG. 1b, it is also possible to use the other side of applicator 6. Positioning ring 5 can be used to position applicator 6 in its other position. Cylindrical part 7 of the applicator comes into contact with the ointment or cream to be applied. This latter position of applicator 6 is particularly suitable for applying the ointment or cream in a body cavity such as the anus.

A container can be used instead of tube 1 described hereinabove, wherein the container has rigid walls and is filled with, in addition to the spreadable composition, a propellant gas for propelling the spreadable substance to the outside. Such a container 11 is shown in FIG. 2. This container 11 is provided with rigid walls 12 and the container is sealed on its top side in a manner known in the art by a top wall 12 in which a controllable valve 13 is placed. This exemplary embodiment is provided with an internal wall 14 of flexible material which serves as separation between the propellant gas, which is situated on the outside of this substantially bag-like wall 14, and the spreadable composition, which is situated on the inside of the wall 14. It must be ensured here that the inner side of wall 14 connects to the valve so that when the valve is operated the spreadable composition flows to the outside. Attachment 15 is used for positioning the applicator. Attachment 15 functions as a positioning element and is mounted releasably on the top wall of the container. This attachment is adapted to position both the spherical part 17 and the sharp part 18 of applicator 16. The releaseability serves to enable cleaning of the attachment.

It is important here that the positioning allows sufficient space for the cream or the ointment to be able to reach the applicator and enter into contact with the applicator. A certain degree of play may be necessary for this purpose. The sealed or impermeable character of the applicator is further of importance here, on the one hand, to ensure that the cream or the ointment does not penetrate into the applicator, but also to enable proper cleaning of the applicator in the case of repeated use.

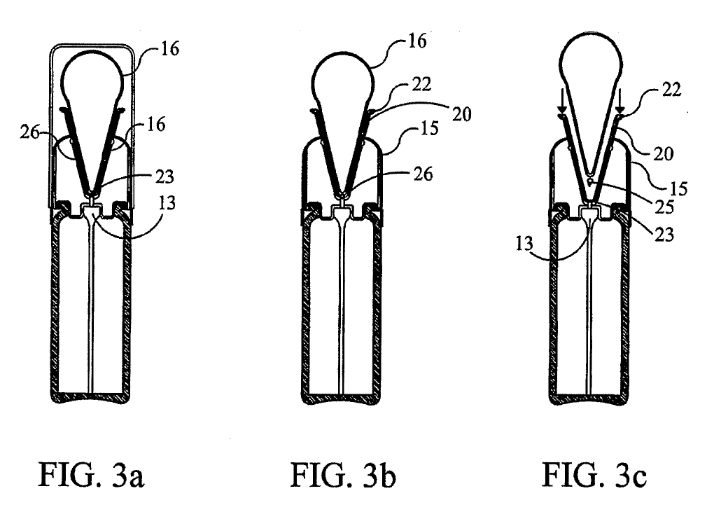

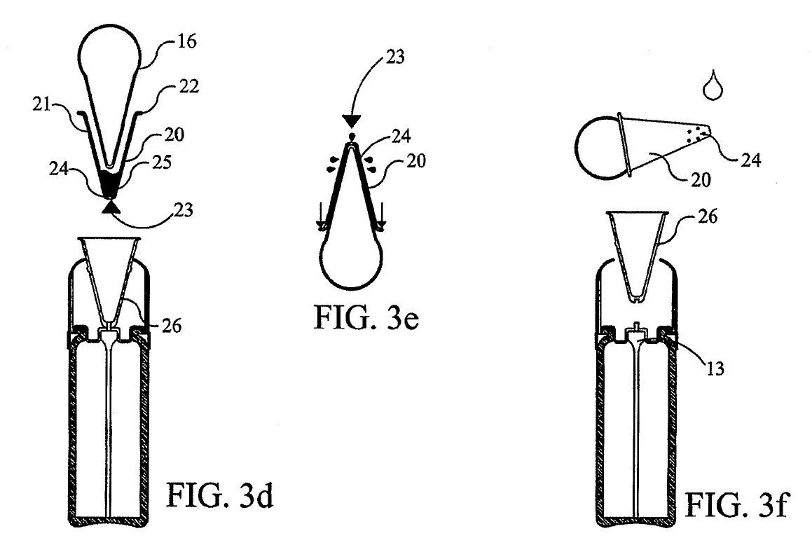

The exemplary embodiment shown in FIGS. 3a-3f differs from the foregoing exemplary embodiment in the different structure of the applicator. The applicator shown in FIGS. 3a-3f is essentially formed by a core 16 which corresponds to the applicator 17 as a whole according to the exemplary embodiment shown in FIG. 2. Around this core 16 is arranged a sleeve 20 which comprises a substantially conical part 21 which is provided at its wide end with a collar 22. The underside of the conical part is provided with a filling opening 23. A number of openings 24 are further placed in conical part 21 in the vicinity of this filling opening 23. The choice of material for sleeve 20 and core 16 is such that core 16 can slide over a limited path inside sleeve 20 wherein, in the manner of a piston, the core closes a cylinder over this path. A space 25 with a variable volume is thus created between the core and the sleeve. It will be apparent that such a variable volume can also be obtained in other ways, for instance, by making use of cylindrical elements. Rigid materials can then suffice for the core and the sleeve.

The container according to this exemplary embodiment differs from the container shown in FIG. 2 in the absence of the flexible wall inside the container. An adapter 26 is also arranged in attachment 15. This adapter 27 serves for better guiding of the applicator when placed on the container. On its top side the adapter 26 is mounted in attachment 15 for movement in an axial direction of the otherwise cylindrical container 11. On its underside the adapter 26 is connected to valve 13 in a manner such that valve 13 of the container is operated when adapter 26 is moved downward. Just as attachment 15, adapter 26 is also removable to enable cleaning. For this purpose, the adapter is provided on its outside with a thickened portion 33 which extends all round and engages releasably under a curved part of attachment 15. A removable cap 27 is arranged for protection of the parts placed on container 11.

The operation of this device will now be described. Starting from the state shown in FIG. 3a in which cap 27 is situated on container 11, cap 27 is first removed. The state shown in FIG. 3b is then obtained. Applicator 16, 20 is now accessible.

By pressing on collar 22 of sleeve 20, the assembly of sleeve 20, core 16 and adapter 27 is moved downward. This state is shown in FIG. 3c. Valve 13 is operated by moving adapter 26 downward whereby valve 13 opens and spreadable composition leaves valve 13 from the container. During this process the spreadable composition fills space 25 between core 16 and sleeve 20 wherein core 16 is forced upward. When sufficient spreadable composition is present in space 25 the user stops pressing sleeve 20.

The applicator in the form of the assembly of sleeve 20 and core 16 can then be taken out, as shown in FIG. 3d. The removed assembly can subsequently be brought to the location on the body or into the body cavity where the spreadable composition must be applied. By moving sleeve 20 to the thickened part 17 of core 16 the space 25 between sleeve 20 and thickened part 17 is reduced, whereby the material present in this space 25 is forced outward through openings 23, 24. This is shown in FIG. 3e.

Applicator 16, 20 can then be cleaned, as can the adapter, for instance, under the hot tap as shown in FIG. 3f. The components can subsequently be replaced on container 11.

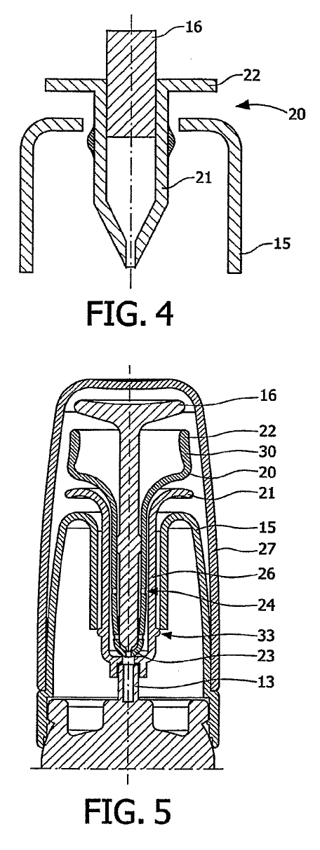

FIG. 4 shows a variant of the exemplary embodiment shown in FIGS. 3a-3f in more detail. This variant differs due to the presence of a cylindrical core 16 which is movable in the cylindrical space in sleeve 20 wherein a good seal between core 16 and sleeve 20 is ensured. This is in contrast to the exemplary embodiment shown in FIGS. 3a-3f wherein the seal is obtained by deforming the substantially conical sleeve 20. In the present exemplary embodiment, sleeve 20 is conical only in the vicinity of filling opening 23. The core is further provided with a scale division 28. It is hereby possible to properly determine the dosage of the spreadable composition.

In the exemplary embodiments described hereinabove, there is always a sleeve in which a piston is displaceable for the purpose of obtaining a space with a variable volume. It is otherwise also possible to obtain a variable volume by making use of a bellows-like applicator wherein a hollow core is used and wherein the cavity of the core connects to a bellows with a variable volume. The tip of the core can also be used to operate the valve of the container. When the valve is operated, the bellows will be filled with spreadable composition; and when the volume of the bellows is reduced, the composition will flow out of openings arranged in the core.

The fourth exemplary embodiment shown in FIG. 5 differs from the exemplary embodiment according to FIGS. 3a-3f in the cylindrical design of the components. The applicator is hereby provided with a cylindrical space, and core 16 will move inside sleeve 20 in the manner of a piston so that the volume of space 25 is reduced by moving core 16 toward sleeve 20, and the spreadable material present in the space is urged out through openings 23, 24 present in the sleeve. In this exemplary embodiment, sleeve 20 further transposes directly into collar 30 so that the contamination of the space between collar 30 and core 20 present in the previous exemplary embodiment is prevented.

It is also possible to arrange two grooves in sleeve 21, not shown in the drawing, which extend in an axial direction of collar 22 to a position a certain distance from the underside of sleeve 21. As a result of the presence of the grooves, the upper part of sleeve 21 can be compressed. This compressibility is used to fixedly clamp core 16 after filling of the applicator during removal from the valve. There is after all then the danger of the content of the space between sleeve 21 and core 16 being suctioned away. A reduction in volume is prevented by fixedly clamping core 16. In order to facilitate the clamping of core 16, the collar 22, which is also divided into two parts by the grooves, is connected to a cylindrical part 30 placed under collar 22. This cylindrical part 30 is also divided into two parts by the grooves to enable compressing of the two resulting pieces of part 30.

It will be apparent that the measures shown in diverse embodiments can be combined. Numerous variations of the exemplary embodiments shown here can, of course, be applied within the scope of the present disclosure.

1. A device for applying a spreadable composition to a part of the human or animal body, comprising:
a) a container containing the spreadable composition;
b) an applicator for the spreadable composition;
c) means for urging the spreadable composition out of the container and into contact with the applicator; and
d) positioning means connected to the container for positioning the applicator in a position in which the composition urged out of the container can be brought into contact with the applicator.

2. The device of claim 1, wherein the container is provided with an outflow opening and the positioning means are adapted to position the applicator in a position in the vicinity of the outflow opening, in the direction of movement of the spreadable composition leaving the outflow opening.

3. The device of claim 1, wherein the container has an internal volume which can be reduced by external forces acting on the container.

4. The device of claim 3, wherein the container is formed by a tube.

5. The device of claim 1, wherein the container has a rigid outer wall and the container comprises a propellant gas in addition to the spreadable composition.

6. The device of claim 5, wherein the container is provided with an internal movable wall, on one side of which the spreadable composition is present and on the other side of which the propellant gas is present.

7. The device of claim 5, wherein the container is provided with a valve which can be operated by the applicator positioned by the positioning means.

8. The device of claim 7, wherein the valve can only be operated by an applicator positioned by the positioning means.

9. The device of claim 1, wherein the applicator is provided with a core with a closed outer wall, said core is for contact with the spreadable composition.

10. The device of claim 9, wherein the core has a first and second part of a mutually different form, wherein both parts can be placed in the positioning means and both parts are adapted to come into contact with the spreadable composition.

11. The device of claim 9, wherein the core is rotation-symmetrical.

12. The device of claim 9, wherein a sleeve is arranged around the core.

13. The device of claim 12, wherein the sleeve is provided with openings for applying the spreadable composition through said openings.

14. The device of claim 12, wherein the sleeve is movable in an axial direction relative to the core.

15. The device of claim 14, wherein the sleeve is provided with a collar extending substantially in a radial direction.

16. The device of claim 12, wherein the sleeve is adapted to be closed internally by the core over a substantial part of the path of movement.

17. The device of claim 16, wherein the interior of the sleeve is cylindrical.

18. The device of claim 16, wherein the sleeve is deformable.

19. The device of claim 14, wherein the sleeve is provided on the end remote from the core with a filling opening for filling the space between the core and the sleeve with the spreadable composition.

20. The device of claim 14, wherein the core is hollow and is provided with a filling opening for filling the space between the core and the sleeve with the spreadable composition.

21. The device of claim 19, wherein the filling opening is adapted to allow the spreadable composition to flow outside when the volume of the space between the sleeve and the core is reduced.

22. The device of claim 16, wherein a scale division is arranged on the core.

23. The device of claim 20, wherein the sleeve and the core are adapted to displace the core relative to the sleeve when the space between the core and the sleeve is filled through the filling opening arranged either in the sleeve or in the core.

24. The device of claim 23, wherein the sleeve is adapted to operate the valve of the container.

25. The device of claim 24, wherein the filling opening in the sleeve is adapted to engage on an operating element of the valve.

26. The device of claim 1, wherein the positioning means are adapted to fix the sleeve by means of a snap connection.

27. The device of claim 26, wherein the snap connection is adapted to allow a movement of the sleeve in axial direction relative to the positioning means.

28. The device of claim 12, wherein the sleeve is replaceably coupled to the core.

29. The device of claim 12, wherein the core is arrestable in the sleeve.

30. The device of claim 29, wherein at least a part of the sleeve is compressible, and the core is arrested in the sleeve when the sleeve is compressed.

31. The device of claim 30, wherein the sleeve is provided with two notches extending on either side of the cavity.

32. The device of claim 1, wherein the applicator is adapted to apply the spreadable composition in a body cavity.

33. The device of claim 32, wherein the applicator is adapted to apply the spreadable composition in the anus.

34. The device of claim 1, wherein the container contains a spreadable composition for use with said device to apply said spreadable composition to a part of the human or animal body.

35. The device of claim 1, wherein the device comprises an applicator for applying a spreadable composition to a part of the human or animal body.

36. A method for applying a spreadable composition to a part of the human or animal body, the method comprising:
a) carrying the spreadable composition out of a container and into contact with an applicator; and
b) applying the spreadable composition from the applicator onto the part of a body, wherein the applicator is placed by positioning means connected to
the container into a position in which the composition urged from the container can be brought into contact with the applicator.

37. The method of claim 36, wherein the spreadable composition is carried out of the container into a space formed in the applicator.

38. The method of claim 37, wherein the spreadable composition flows out of the container into the space formed in the applicator through the operation of a valve of the container by the applicator.

39. The method of claim 37, wherein the spreadable composition is urged from the applicator to the body by reducing the volume of the cavity formed in the applicator.

40. The method of claim 39, wherein the spreadable substance is carried at least partially through the filling opening into and out of the space.

41. The method of claim 36, wherein the volume of the space is read during filling of the space between core and sleeve.
